(19) United States
(12) Patent Application Publication
(10) Pub. No.: US 2011/0313044 A1
(43) Pub. Date: Dec. 22, 2011

(54) Polymorphs of Suberoylanilide Hydroxamic Acid

(75) Inventors: Xavier Vila Tusell, Hostalric (ES); Cristina Puigjaner Vallet, Barcelona (ES); Rafel Prohens López, Llinars del Valles (ES); Anna Portell Bueso, Barcelona (ES); Ismael Valverde Martin, Mollet del Valles (ES)

(73) Assignee: URQUIMA S.A., Barcelona (ES)

(21) Appl. No.: 13/161,763

(22) Filed: Jun. 16, 2011

Related U.S. Application Data
(60) Provisional application No. 61/356,249, filed on Jun. 18, 2010.
(60) Provisional application No. 61/367,647, filed on Jul. 26, 2010.

(51) Int. Cl. A61K 31/167; A61P 35/00

(52) U.S. Cl. 514/575

(57) ABSTRACT

The present invention is directed to a novel form of suberoylanilide hydroxamic acid and the processes for its preparation.

This application claims priority from U.S. Provisional Application Ser. No. 61/367,647, filed Jul. 26, 2010 and U.S. Provisional Application Ser. No. 61/356,249, filed Jun. 18, 2010. These prior applications are incorporated herein by reference.

### FIELD OF THE INVENTION

The present invention relates to a novel crystalline composition comprising suberoylanilide hydroxamic acid (SAHA) and ammonia and a process for its preparation. The present invention also provides a method for combining suberoylanilide hydroxamic acid and ammonia to obtain pure crystalline Form α of suberoylanilide hydroxamic acid.

### BACKGROUND OF THE INVENTION

Vorinostat, also called suberoylanilide hydroxamic acid (SAHA) or N-hydroxy-N′-phenyloctanediamide, is represented by the structural formula (I).

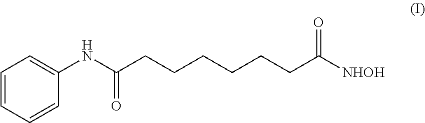

Vorinostat, a histone deacetylase (HDAC) inhibitor, is currently marketed with trade name Zolinza® for the treatment of cutaneous T-cell lymphoma (CTCL), a type of skin cancer. It is administered to treat patients who have a progressive, persistent or recurrent disease and who are on or have had two systemic therapies. Vorinostat is thought to be useful for selectively inducing terminal differentiation of neoplastic cells and thereby inhibiting proliferation of such cells under suitable conditions. Zolinza® is supplied as white, opaque, hard gelatine capsules with a strength of 100 mg. The recommended dose is 400 mg orally once daily with food.

Vorinostat and different synthetic strategies for its preparation are reported in U.S. Pat. No. 5,369,108.

Stowell, J. et al., in J. Med. Chem. 38(8) 1411-13, 1995 and Mai, A. et al., in Organic Preparations and Procedures International, 33(4), 391-394, 2001 describe different methods for synthesizing Vorinostat.

Processes for the preparation of Vorinostat, and its Form I polymorph, have been disclosed in U.S. Pat. No. 7,456,219 and patent application WO 2006/127319. Moreover, in these patents have been reproduced procedures of obtention of Vorinostat disclosed in the prior art. Designated Form II, Form III, Form IV and Form V are obtained according to prior art procedures, including those of U.S. Pat. No. 5,369,108; Mai, A. et al., Organic Preparations and Procedures International, 33(4), 391-394, 2001, and Stowell, J. et al., J. Med. Chem. 38(8) 1411-13, 1995. Form I, Form II, Form III, Form IV and Form V are characterized by powder X-ray diffraction (PXRD) and Differential Scanning calorimetry (DSC).

Research Disclosure Publication No. 547,018 published Oct. 15, 2009, provides a novel crystalline form of SAHA, Form α, characterized by PXRD, DSC and Thermal Gravimetric Analysis (TGA), and a procedure for its preparation.

### SUMMARY OF THE INVENTION

The present invention is directed to a novel crystalline composition comprising suberoylanilide hydroxamic acid (SAHA) and ammonia. Preferably, the composition consists essentially of SHA and ammonia.

In another embodiment, the novel crystalline composition has a mole ratio 2:1 of SAHA:ammonia.

In another embodiment, there is provided a process for preparing a crystalline composition consisting essentially of suberoylanilide hydroxamic acid and ammonia, comprising the step of putting in contact suberoylanilide hydroxamic acid with ammonia until crystal growth is promoted.

In another aspect, there is provided a process for the preparation of a crystalline composition comprising suberoylanilide hydroxamic acid and ammonia comprising; a) forming a solution of suberoylanilide hydroxamic acid in an organic solvent; b) promoting crystal growth by adding ammonia; and c) collecting the crystals.

The present invention is also directed to a pure crystalline Form α of suberoylanilide hydroxamic acid obtainable by heating a crystalline composition comprising suberoylanilide hydroxamic acid and ammonia at a temperature within the range of from about 115° C. up to about 150° C.

In another embodiment, there is provided a process for preparing a stable and pure crystalline Form α of suberoylanilide hydroxamic acid, which comprises the step of heating a composition comprising suberoylanilide hydroxamic acid SAHA and ammonia at a temperature within the range of from about 115° C. up to about 150° C.

The present invention is also directed to a stable and crystalline pure Form α of suberoylanilide hydroxamic acid having a powder X-ray diffraction pattern including characteristic peaks at about 9.3, 10.7, 14.5, 14.9, 15.4, 17.7, 19.9, 21.3, 21.5 and 23.3 degrees 2θ, wherein the X-ray diffraction is measured with a Copper X-ray source; and further characterized by DSC thermogram having an endothermic peak at about 162.5±2.0° C.

In another aspect, there is provided a pharmaceutical composition which comprises a therapeutically effective amount of stable and crystalline pure Form α of SAHA, together with one or more suitable pharmaceutically acceptable excipients or carriers.

There is also provided use of stable and crystalline pure Form α of SAHA in the treatment of cutaneous T-cell lymphoma (CTCL) and for selectively inducing terminal differentiation of neoplastic cells and thereby inhibiting proliferation of such cells under suitable conditions.

The foregoing and other objects, features and advantages of the invention will be apparent from the following more particular description of preferred embodiments of the invention, as illustrated in the accompanying drawings.

### BRIEF DESCRIPTION OF THE DRAWINGS

The drawings provided are not necessarily to scale, emphasis instead being placed upon illustrating the principles of the invention. Like reference characters refer to the same parts throughout different views.

FIG. 1 shows a comparative x-ray diffractogram for crystalline forms of suberoylanilide hydroxamic acid Form I, Form II, Form III and Form α.

FIG. 2 shows a comparative DSC thermogram for crystalline forms of suberoylanilide hydroxamic acid Form I, Form II, Form III and Form α.

FIG. 3 shows x-ray diffractogram for a crystalline composition consisting essentially of suberoylanilide hydroxamic acid and ammonia.

FIG. 4 shows IR for a crystalline composition consisting essentially of suberoylanilide hydroxamic acid and ammonia.

FIG. 5 shows DSC thermogram for a crystalline composition consisting essentially of suberoylanilide hydroxamic acid and ammonia.

FIG. 6 shows TGA analysis for a crystalline composition consisting essentially of suberoylanilide hydroxamic acid and ammonia.

FIG. 7 shows x-ray diffractogram for pure crystalline Form α of suberoylanilide hydroxamic acid.

FIG. 8 shows DSC thermogram for pure crystalline Form α of suberoylanilide hydroxamic acid.

FIG. 9 shows TGA analysis for pure crystalline Form α of suberoylanilide hydroxamic acid.

FIG. 10 shows IR for pure crystalline Form α of suberoylanilide hydroxamic acid.

### DETAILED DESCRIPTION OF THE INVENTION

The present invention is directed to a novel isolated polymorph of suberoylanilide hydroxamic acid (SAHA) characterized by specific powder X-ray diffraction, TGA, IR, and DSC thermogram and to methods of making this polymorph. The present invention also provides pharmaceutical compositions which comprise a novel polymorph of suberoylanilide hydroxamic and one or more excipients.

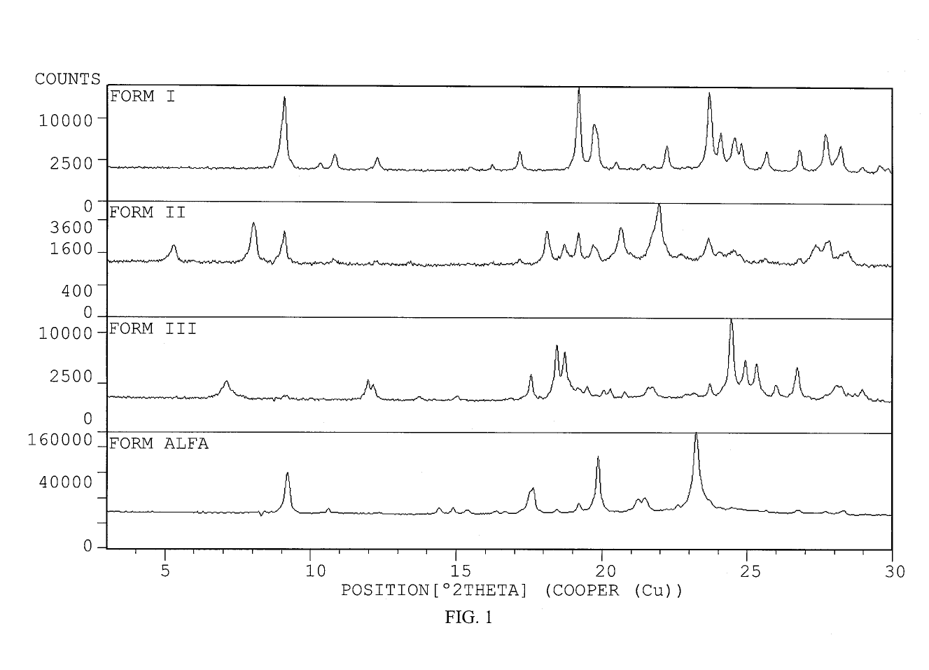

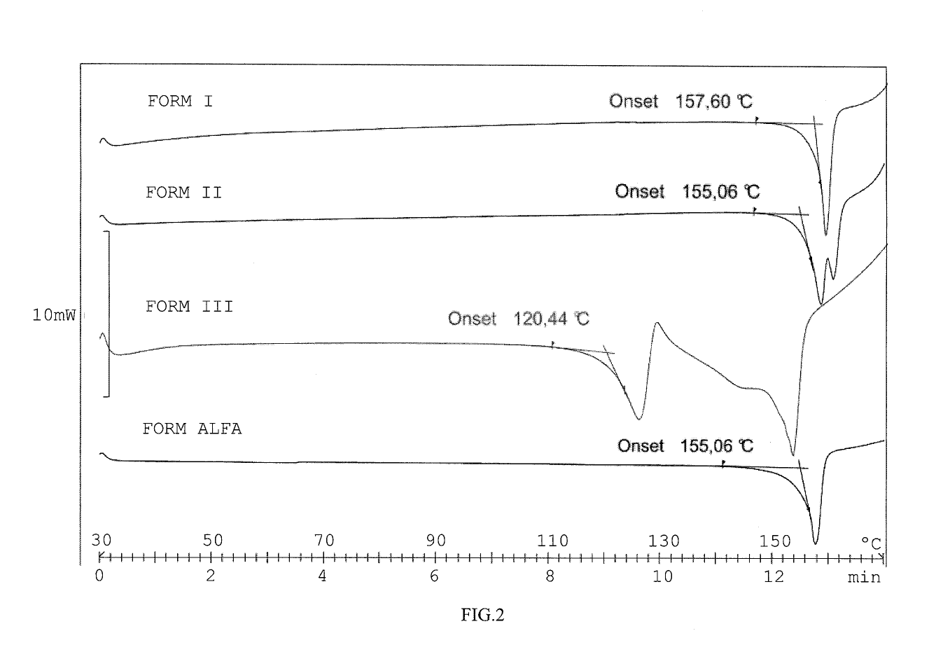

Experiments have been carried out with the objective of obtaining crystalline forms disclosed in prior art. The crystalline products obtained have been characterised by XPRD (FIG. 1 and Table 1) and DSC (FIG. 2). Discussion of the results follows: Form I, disclosed in U.S. Pat. No. 7,456,219, is an anhydrate polymorph stable at room temperature.

Form II, disclosed in U.S. Pat. No. 7,456,219, is an anhydrate polymorph. The product obtained contains Form I. Apparently, Form II is unstable at room temperature, degrading spontaneously into Form I.
Form III, disclosed in U.S. Pat. No. 7,456,219, is an anhydrate polymorph stable at room temperature. Form III is obtained with different ratios of Form I.
Form IV and Form V, according to characterization disclosed in U.S. Pat. No. 7,456,219, could be considered by the skilled person in the art as a blend of Form I with at least one second species.
Form α, described in Research Disclosure Publication, No. 547,018, is an anhydrate polymorph stable at room temperature. However, procedures disclosed in this publication describe the obtainment of an impure Form α present in combination with Form I and/or Form II. PXRD submitted in this Publication presents a peak at about 19.2θ, which is a characteristic peak of polymorph Form I.

TABLE 1

| Form I | | Form II | | Form III | | Form α | |
|---|---|---|---|---|---|---|---|
| | Rel. | | Rel. | | Rel. | | Rel. |
| Pos. | Int. | Pos. | Int. | Pos. | Int. | Pos. | Int. |
| [°2Th.] | [%] | [°2Th.] | [%] | [°2Th.] | [%] | [°2Th.] | [%] |
| 9.1 | 79.5 | 5.3 | 21.1 | 7.1 | 11.8 | 9.2 | 38.2 |
| 10.3 | 3.4 | 8.0 | 57.9 | 9.2 | 1.9 | 10.6 | 2.8 |
| 10.8 | 9.6 | 9.1 | 43.6 | 12.0 | 13.6 | 14.4 | 3.7 |
| 12.3 | 7.4 | 18.1 | 45.7 | 15.0 | 2.2 | 14.9 | 3.9 |
| 17.2 | 12.4 | 18.7 | 24.7 | 17.6 | 18.8 | 15.4 | 2.2 |
| 19.2 | 100.0 | 19.2 | 43.1 | 18.4 | 54.0 | 16.4 | 1.4 |
| 19.7 | 40.9 | 19.7 | 22.3 | 18.7 | 44.2 | 17.6 | 19.8 |
| 20.5 | 5.2 | 20.6 | 51.9 | 19.2 | 8.2 | 18.4 | 1.8 |
| 22.2 | 18.6 | 21.9 | 100.0 | 19.5 | 9.5 | 19.2 | 5.9 |
| 23.7 | 91.1 | 23.7 | 34.4 | 20.1 | 6.7 | 19.9 | 59.9 |
| 24.1 | 32.3 | 24.6 | 15.3 | 20.3 | 8.0 | 21.2 | 8.3 |
| 24.6 | 27.4 | 27.3 | 23.5 | 20.8 | 5.6 | 21.5 | 9.3 |
| 24.8 | 21.5 | 27.8 | 31.2 | 21.6 | 8.4 | 22.6 | 3.7 |
| 25.6 | 14.3 | 28.4 | 16.4 | 21.7 | 9.0 | 23.2 | 100.0 |
| 26.8 | 15.9 | | | 23.7 | 12.2 | 26.7 | 1.5 |
| 27.7 | 31.8 | | | 24.4 | 100.0 | 27.7 | 1.4 |
| 28.2 | 19.7 | | | 24.9 | 34.9 | 28.3 | 2.5 |
| 29.5 | 5.2 | | | 25.3 | 30.8 | | |
| | | | | 26.0 | 11.7 | | |
| | | | | 26.7 | 28.3 | | |
| | | | | 28.1 | 11.6 | | |
| | | | | 28.2 | 10.5 | | |
| | | | | 28.9 | 8.9 | | |

Therefore, pure crystalline Form α of suberoylanilide hydroxamic acid has not previously been obtained or described.

A disadvantage with the previously disclosed polymorphic forms of suberoylanilide hydroxamic acid is that they are usually obtained as a blend of more than one crystalline form.

Polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different X-ray diffraction peaks. Since the solubility of each polymorph can vary, identifying the existence of pharmaceutical polymorphs is essential for providing pharmaceuticals with predictable solubility profiles. Polymorphic forms of a compound can be distinguished by X-ray diffraction spectroscopy and by other methods such as infrared spectrometry. Additionally, the properties of polymorphic forms of the same active pharmaceutical ingredient are well known in the pharmaceutical art to have an effect on the manufacture of drug product compositions comprising the active pharmaceutical ingredient. For example, the solubility, stability, flowability, tractability and compressibility of the active pharmaceutical ingredient, as well as the safety and efficacy of the drug product, can be dependent on the crystalline from.

The discovery of new polymorphic forms of a pharmaceutically useful compound provides a new opportunity to improve the performance characteristics of the pharmaceutical product. It also adds to the choice that a formulation scientist has available for designing, for example, a pharmaceutical dosage form of a drug with a targeted release profile or other desired characteristics.

To prepare pharmaceutical compositions containing suberoylanilide hydroxamic acid for administration to mammals in accordance with exacting health registration requirements of the U.S. and international health registration authorities, e.g. the FDA's Good Manufacturing Practices requirements, there is a need to produce suberoylanilide hydroxamic acid in as pure a form as possible, especially a form having constant physical properties.

Hence, there is a need in the art for novel, stable and pure crystalline polymorphic form of SAHA.

We now surprisingly and unexpectedly have obtained a novel crystalline composition consisting essentially of suberoylanilide hydroxamic acid and ammonia, from which can be obtained a stable and pure crystalline Form α of suberoylanilide hydroxamic acid. Furthermore, the disclosed method of obtaining pure crystalline Form α via an intermediate comprising suberoylanilide hydroxamic acid and ammonia, results in a new process which reduces substantially extra processing steps for the purification of the product, reducing the cost and the time to manufacture the product.

Hence, compared with SAHA's polymorphic forms disclosed in the prior art, Form II and Form III, crystalline pure Form α is obtainable using a crystalline composition comprising suberoylanilide hydroxamic acid as an intermediate.

Moreover, solubility tests performed in methanol and HCl 2M show the higher solubility of crystalline pure Form α versus Form I.

In addition, the improved solubility showed by crystalline pure Form α in a hygroscopicity test performed reveals that this polymorph is a non-hygroscopic product.

According to one aspect of the present invention, there is provided a novel crystalline composition comprising suberoylanilide hydroxamic acid and ammonia, characterized by at least one, and preferably all, of the following properties:

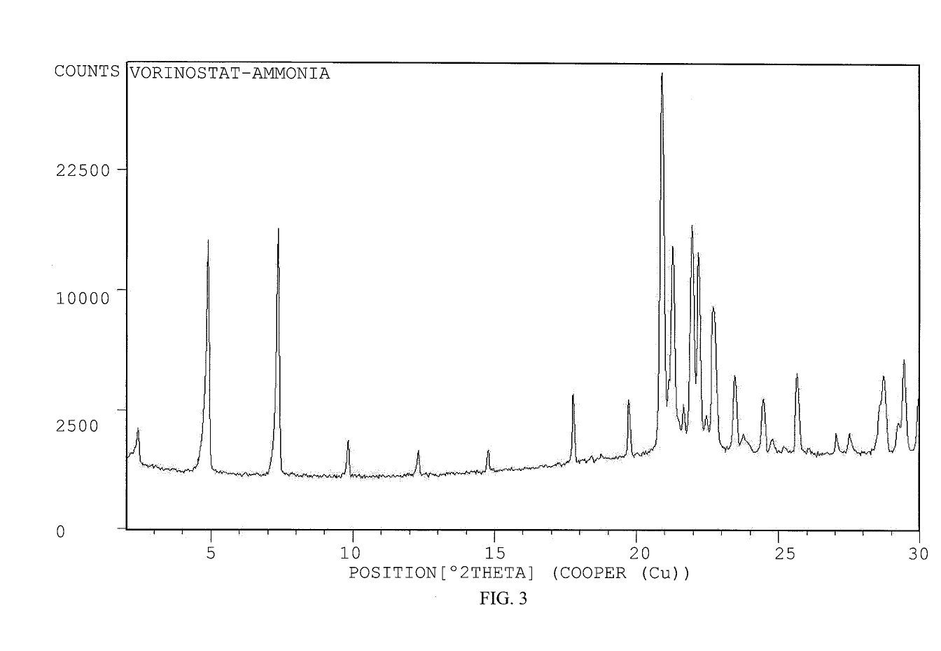

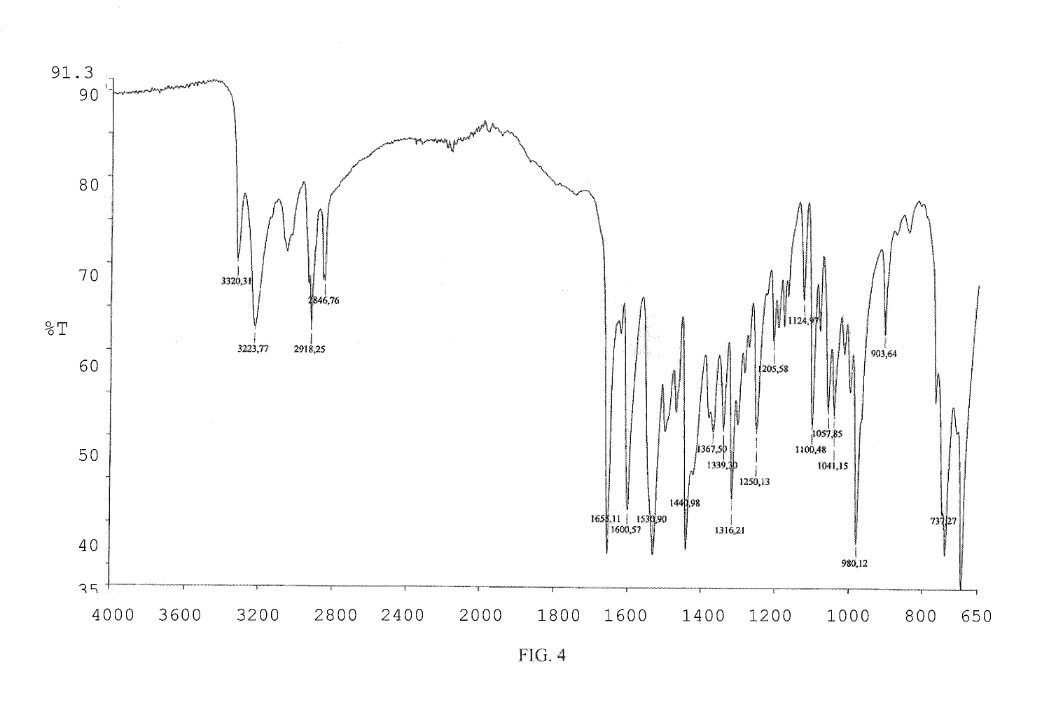

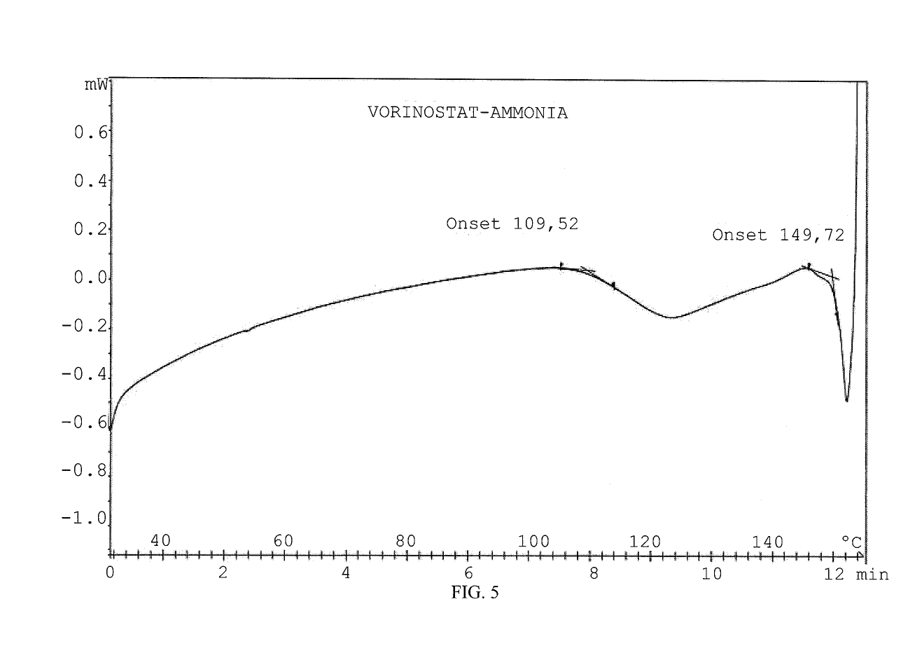

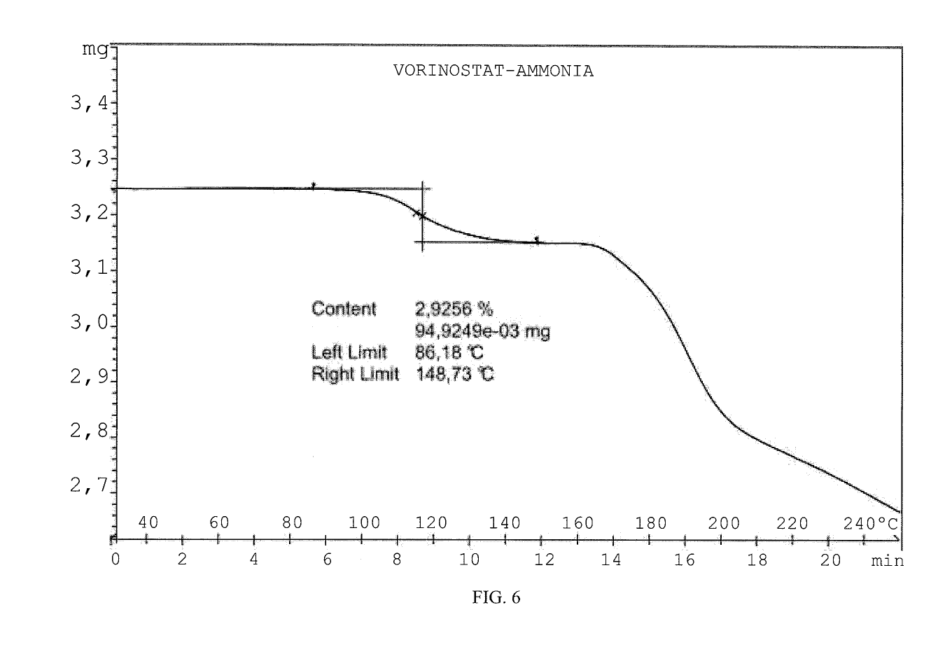

i) a powder X-ray diffraction pattern substantially in accordance with FIG. 3;
ii) a powder X-ray diffraction pattern having peaks at about 2.4, 4.9, 7.4, 20.9, 21.4, 21.9, 22.2 and 22.7 degrees 2θ;
iii) a DSC thermogram substantially as depicted in FIG. 5;
iv) a DSC thermogram having two endothermic peaks;
v) an IR Spectrum substantially in accordance with FIG. 4;
vi) an IR Spectrum having absorption bands at about 3320, 3224, 3051, 1601 and 980 (cm⁻¹); or
vii) a TGA substantially in accordance with FIG. 6.

DSC of the crystalline composition comprising suberoylanilide hydroxamic acid and ammonia shows a first endothermic phenomenon from about 109° C. due to the evaporation of NH₃ followed by a second endothermic phenomenon at about 150° C. attributable to the melting of SAHA after losing ammonia. The evaporation of ammonia is a kinetic phenomenon and it is possible to observe it at different temperatures, therefore it is not an exact value useful to characterize a compound (the parameter which characterizes an anhydrous form by DSC is its melting onset). On the other hand, the melting observed after losing ammonia can also be observed at different temperatures because depending on how ammonia evaporates, different SAHA crystal forms can be obtained.

In a preferred embodiment the new crystalline composition of suberoylanilide hydroxamic acid and ammonia has a mole ratio 2:1 of SAHA:ammonia. Mole ratio was confirmed by Quantiative Elemental Analysis according to the values of Table 2 and TGA (FIG. 6).

TABLE 2

| | % C | % H | % N |
|---|---|---|---|
| Theoretical values (1:1) | 59.77 | 8.24 | 14.94 |
| Theoretical values (2:1) | 61.63 | 7.94 | 12.83 |
| Found values | 61.29 | 8.42 | 12.86 |

According to another aspect of the present invention, the invention provides a process for preparing a crystalline composition comprising suberoylanilide hydroxamic acid and ammonia, comprising the step of contacting suberoylanilide hydroxamic acid with ammonia until crystal growth is promoted. Exemplary ammonia sources include, but are not limited to, NH₃ gas, NH₃ aqueous solution and organic solvent solutions of NH₃. Exemplary methods of contacting SAHA with ammonia include treating a solution of SAHA with a solution of ammonia or with ammonia gas; treating a suspension/slurry of SAHA with a solution of ammonia or with ammonia gas; and treating solid SAHA with a solution of a ammonia or with ammonia gas.

An exemplary method for obtaining a slurry or a suspension comprises contacting SAHA with a solvent in which SAHA has a low solubility. Examples of suitable solvents include THF, dioxane, acetone or acetonitrile.

According to another aspect of the present invention, there is provided a process for the preparation of a novel crystalline composition comprising SAHA and ammonia, which comprises:
a. forming a solution of suberoylanilide hydroxamic acid in a organic solvent;
b. promoting crystal growth by adding ammonia; and
c. collecting the crystals.

The organic solvent used in step a) can be selected from alcohols, ketones, nitriles, cyclic ethers, aliphatic ethers and mixtures thereof. Exemplary alcohol solvents include, but are not limited to, C1 to C6 straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof. Exemplary ketone solvents include, but are not limited to, acetone, methyl ethyl ketone, methyl isobutyl ketone, methyl tert-butyl ketone and the like, and mixtures thereof.

Exemplary nitrile solvents include, but are not limited to, acetonitrile, propionitrile and the like, and mixtures thereof. Exemplary cyclic ether solvents include, but are not limited to, tetrahydrofuran, dioxane, and the like, and mixtures thereof. Exemplary aliphatic ether solvents include, but are not limited to, diethyl ether, diisopropyl ether, monoglyme, diglyme and the like, and mixtures thereof.

Preferably, the solvent is selected from methanol, ethanol, isopropanol, DMF, DMSO and mixtures thereof.

Step a) comprises either forming a solution of SAHA by dissolving SAHA in an organic solvent, or using an existing solution of SAHA from a previous reaction step.

Preferably, SAHA is dissolved in the organic solvent at a temperature from about 0° C. to about the reflux temperature of the solvent used, more preferably from about 10° C. to about 60° C., and still more preferably from about 20° C. to about 40° C.

The solution in step a) may be prepared by reacting a suberanilic acid derivative with freshly prepared hydroxilamine, in accordance with the procedure described in Mai, A. et al., in Organic Preparations and Procedures International, 33(4), 391-394, 2001.

Step b) of promoting crystal growth can be carried out by adding ammonia as a gas or in a solution. Ammonia addition is preferably done at a temperature from about 0° C. to about 45° C., more preferably from about 10° C. to about 35° C., and still more preferably from about 20° C. to about 30° C. Exemplary solvents for the ammonia solution include, but are not limited to, methanol, ethanol, isopropanol, water and dioxane and the like, and mixtures thereof.

Preferably, the crystallization is carried out by cooling the solution at a temperature of below about 30° C. for at least 10 minutes, more preferably at about 0° C. to about 25° C. for a period of from about 1 hour to about 10 hours, and still more preferably from about 2° C. to about 5° C. for a period of from about 2 hours to about 3 hours.

Step c) of collecting the crystals can be carried out by conventional techniques known in the art such as filtration, filtration under vacuum, decantation, centrifugation, or a combination thereof. Collected crystals can be washed using any suitable solvent. Preferably, the solvent is selected from acetone, acetonitrile and mixtures thereof.

The crystalline composition comprising suberoylanilide hydroxamic acid and ammonia of the present invention is obtained with high yields and elevated richness, i.e. with a purity greater than 99%.

According to another aspect of the present invention, there is provided a stable and pure crystalline Form α of SAHA obtainable by heating of a crystalline composition comprising suberoylanilide hydroxamic acid and ammonia. The heating for this step can be carried out for shorter or longer periods of time depending on the product specification. A preferred range of temperature on heating is from about 115° C. up to about 150° C. A more preferred temperature range is from about 120° C. to about 130° C.

The time necessary for the heating step depends on the batch size. Although a PXRD analysis is possibly the best option to control the transformation of a crystalline suberoylanilide hydroxamic acid and ammonia composition into highly pure crystalline and stable Form α of SAHA, other control standard industrial methods also can be adapted to this purpose. Examples of control standard industrial methods for this goal are Loss-On-Drying, IR and DSC.

In another aspect, provided herein is a process for the preparation of a pure crystalline and stable Form α of SAHA, which comprises the step of heating a suberoylanilide hydroxamic acid and ammonia composition in a range of temperature from about 115° C. up to about 150° C. In a preferred embodiment the heating range of temperature is from about 120° C. to about 130° C.

The suberoylanilide hydroxamic acid and ammonia crystalline composition of the present invention is obtained with high yields and elevated richness, i.e. with a purity greater than 99%. Thermal transformation of suberoylanilide hydroxamic acid and ammonia converts to stable and pure crystalline Form α of SAHA with nearly quantitative yield.

Stable and pure crystalline Form α of SAHA is characterized by at least one, and preferably all, of the following properties:

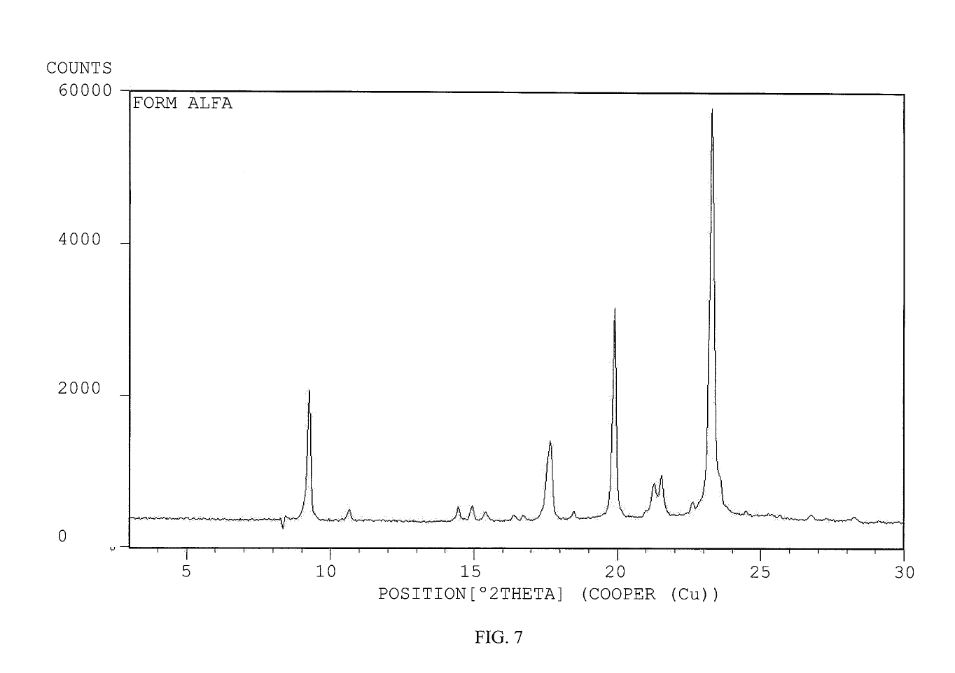

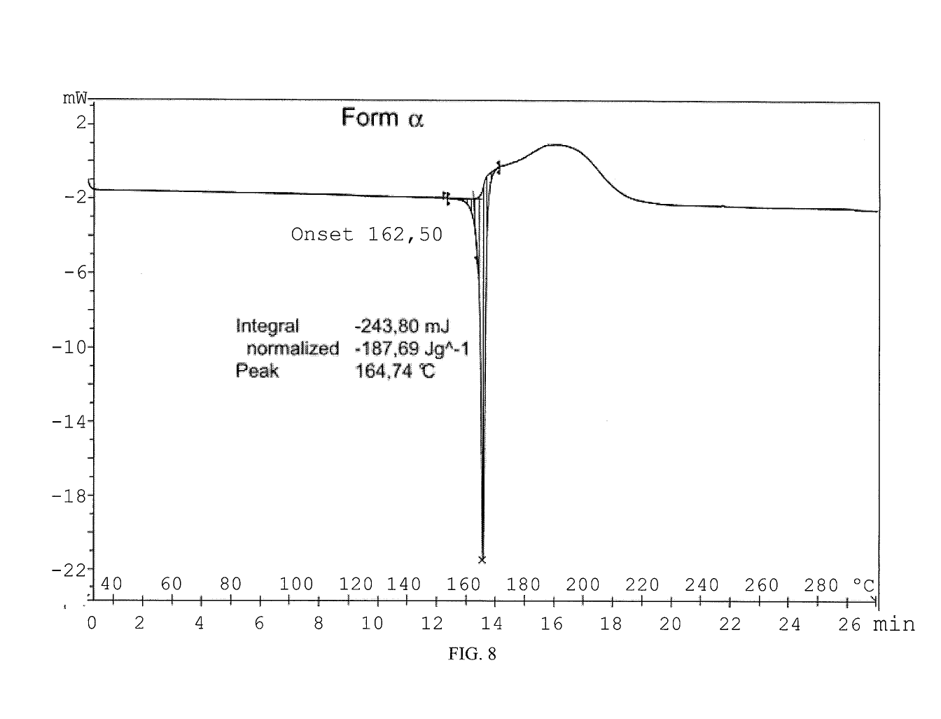

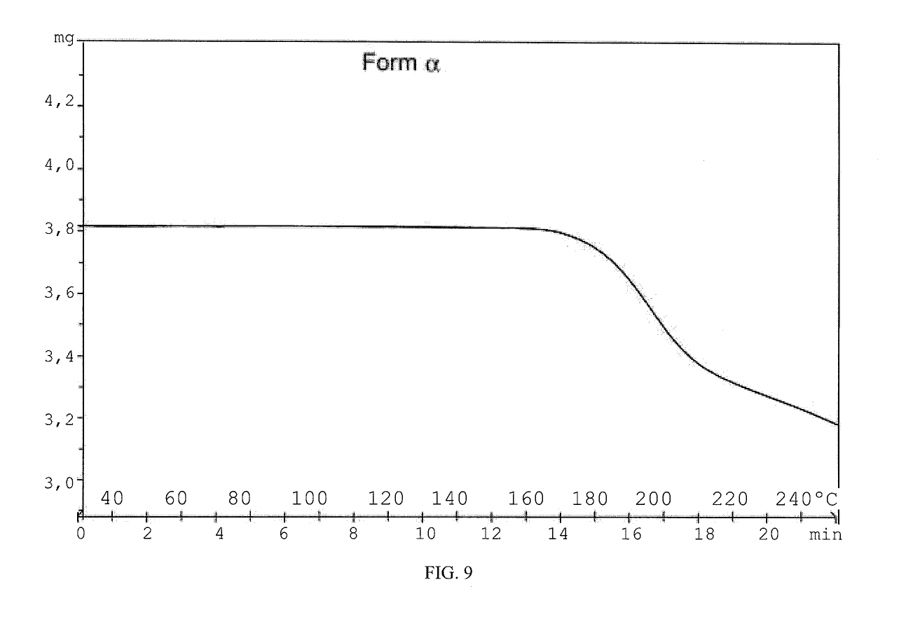

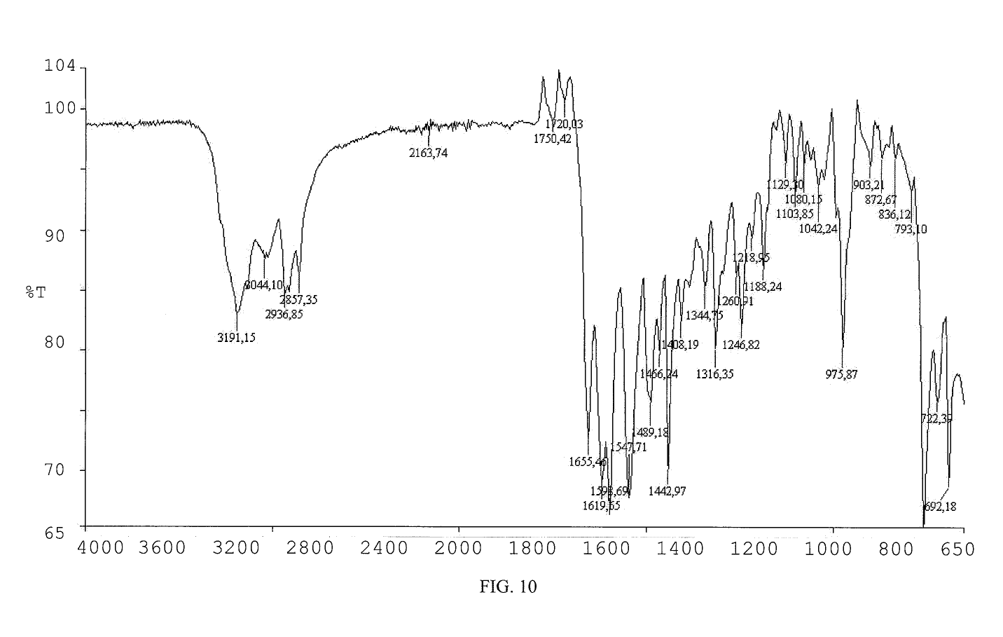

i) a powder X-ray diffraction pattern substantially in accordance with FIG. 7;
ii) a powder X-ray diffraction pattern having peaks at about degrees 2θ substantially as depicted in FIG. 7;
iii) a powder X-ray diffraction pattern having peaks at about degrees 2θ substantially according to Table 3;
iv) DSC thermogram substantially as depicted in FIG. 8;
v) a DSC thermogram having endothermic peak at about 162.5±2.0° C.;
vi) an IR spectrum substantially in accordance with FIG. 10;
vii) an IR spectrum having absorption bands at about 1620, 1489, 1104 and 722 (cm⁻¹); or
viii) a TGA substantially in accordance with FIG. 9.

TABLE 3
Pure Crystalline Form α
(Example 12)

| Pos. [°2Th.] | Rel. Int. [%] |
|---|---|
| 9.3 | 31.8 |
| 10.7 | 2.6 |
| 14.5 | 3.5 |
| 14.9 | 3.9 |
| 15.4 | 2.3 |
| 16.4 | 1.6 |
| 16.7 | 1.4 |
| 17.7 | 19.0 |
| 18.5 | 2.7 |
| 19.9 | 51.5 |
| 21.3 | 9.1 |
| 21.5 | 11.4 |
| 23.3 | 100.0 |

In a preferred embodiment, stable and pure crystalline Form α of SAHA is characterized by at least one, and preferably all, of the properties of above and lacking a PXRD peak at about 19.2 degrees 2θ.

The pharmaceutical compositions of the present invention comprise a therapeutically effective amount of the polymorph Form α of SAHA of the present invention, together with at least one suitable pharmaceutically acceptable excipient or carrier. The product of the present invention can be formulated in accordance with standard pharmaceutical practice. In a preferred embodiment, the pharmaceutical composition of the present invention is an oral composition which comprises pure crystalline Form α of SAHA and one or more suitable pharmaceutically acceptable excipients or carriers. In a most preferred embodiment, the pharmaceutical composition of the present invention is an oral composition which comprises pure crystalline Form α of SAHA and one or more suitable pharmaceutically acceptable excipients or carriers with a strength within the range of about 50 to about 250 mg, preferably of about 100 mg.

The polymorph Form α of SAHA of the present invention is useful in the treatment of patients suffering from cutaneous T-cell lymphoma (CTCL) who have progressive, persistent or recurrent disease and who are undergoing or have had two systemic therapies.

Differential scanning calorimetry (DSC) analyses were carried out by means of a Mettler-Toledo DSC-822e calorimeter. Experimental conditions: aluminum crucibles of 40 μL volume, atmosphere of dry nitrogen with 50 mL/min flow rate, heating rate of 10° C./min. The calorimeter was calibrated with indium of 99.99% purity. Instrument could result in a different thermogram. The present invention is characterized by the thermogram values set forth herein obtained using this DSC Instrument as well as the equivalent thermogram values obtained using other types of DSC instruments.

Thermal Gravimetric Analyses (TGA) were performed on a Mettler-Toledo TGA-851e thermobalance. Experimental conditions: alumina crucibles of 70 μL volume, atmosphere of dry nitrogen with 50 mL/min flow rate, heating rate of 10° C./min.

Pure crystalline Form α of suberoylanilide hydroxamic acid PXRD analyses: The analysed powder samples with Powder X-ray diffraction (PXRD) were placed in Lindemann glass capillaries of 0.5 millimeters of diameter and analysed in a INEL CPS-120 powder diffractometer in transmission Debye-Scherrer geometry, operating at 40 kV and 30 mA with CuKα 1 radiation (λ=1.5406 Å), selected by means of a Ge (111) primary beam monochromator, and a 120° curved position sensitive detector. The useful measured range was between 2 and 35° in 2θ and the acquisition time of 17779 seconds.

Crystalline composition consisting essentially of suberoylanilide hydroxamic acid and ammonia PXRD analyses: The powder samples were introduced in a Lindemann glass capillary of 0.7 millimeters of diameter and analysed in a PANalytical X'Pert PRO MPD θ/θ powder diffractometer of 240 millimetres of radius, in a configuration of convergent beam with a focalizing mirror and a transmission geometry with a spinner glass capillary sample holder, in the following experimental conditions: Cu Kα radiation (1=1.5418 Å), Work power: 45 kV-40 mA, Incident beam slits defining a beam height of 0.4 millimetres. Incident and diffracted beam 0.02 radians Soller slits, PIXcel detector: Active length=3.347°, 2θ scans from 2 to 60° 2θ with a step size of 0.026° 2θ and a measuring time of 200 seconds per step.

The term “about” when used in the context of an amount refers to ±10% of the specified amount. For the purpose of this invention, for X-ray diffraction patterns, depending on the calibration, sample or instrumentation, peaks at 20 can shift up to ±0.2 degrees (error). In one embodiment, all peaks in X-ray diffraction pattern shift up to +0.2 degrees, or up to −0.2 degrees. An X-ray diffraction pattern or peaks within that error is considered the same or substantially similar.

Fourier Transform Infrared-Attenuated Total Reflectance (FT-IR-ATR) sprectra were registered on a Perkin Elmer Spectrum One/100 FT-IR spectrometer with universal attenuated total reflectance (ATR) sampling accessory (SPECTRUM100 with UATR1BOUNCE). Sample is placed on the ATR plate and the measure is carried out in the 650-4000 cm⁻¹ range. The term “IR or IR spectrum/spectra” when used in the context refers to spectra registered in the conditions mentioned above.

The term “crystalline pure” when used in the context refers to a crystalline form of a highly pure product comprising no more than about 1% of another crystalline form of the same product. Crystalline pure SAHA Form α comprises no more than 1% each of crystalline SAHA Form 1 and the SAHA—ammonia species, preferably no more than about 0.5% each of SAHA Form 1 and the SAHA—ammonia species.

The term “room temperature” when used in the context refers to a temperature from about 15° C. to about 30° C.

The term “ammonia” when used in the context refers to NH₃ or a source of NH₃.

The term “stable” when used in the context refers to a physical or chemical stability of a product.

As used herein, “reflux temperature” means the temperature at which the solvent or solvent system refluxes or boils at atmospheric pressure.

The term “species” when used in the context refers to any product with different physical characteristics present in a solid sample.

Throughout the description and the claims the word “comprises” and its variants are not meant to exclude other technical characteristics, additives, components or steps. For skilled persons in the art, other objects, advantages and characteristics of the invention can be deduced in part from the description and partly from the practice of the invention.

### Experimental Details Section

### Example 1

### Preparation of Suberoylanilide Hydroxamic Acid (SAHA)

A solution of suberanilic acid (21.6 g) in THF (432 mL) chilled at 0° C. was added to triethylamine (18.3 mL) and ethyl chloroformate (11.4 mL). The suspension thus formed was stirred at that temperature for 10 min, after that time the suspension was filtered. The filtrate was added to freshly prepared hydroxylamine (hydroxylamine hydrochloride, 10.1 g) (prepared according to J.P. Devlin, J. Chem. Soc. Perkin 1, 846, 1975). Once the addition was finished, the system was allowed to stir for an additional 15 min. After that time the solution was concentrated to dryness, and the residue (29.0 g) was treated with acetonitrile (216 mL) at 80° C. for an hour. The suspension was allowed to reach room temperature, the solid was filtered, dried in the oven at 50° C. under vacuum, and the desired compound was obtained as a white solid (20.6 g, 90% yield, ¹H-NMR (DMSO-d6, 300 MHz): 1.20-1.36 (m, 4H, H-4 and H-5), 1.49 (qn, J=7 Hz, 2H, H-3 or H-6), 1.57 (qn, J=7 Hz, H-3 or H-6), 1.94 (t, J=7.3 Hz, 2H, H-2 or H-7), 2.28 (t, J=7.4 Hz, 2H, H-2 or H-7), 7.01 (t, J=1, 7.4 Hz, 1H, H-4′), 7.27 (t, J=7.9 Hz, 2H, H-3′ and H-5′), 7.58 (d, J=7.6 Hz, 2H, H-2′ and H-6′), 8.67 (bs, 1H, NH), 9.93 (s, 1H, NH), 10.38 (bs, 1H, OH), ¹³C-NMR (DMSO-d6, 75.5 MHz): 25.0 (C-3 and C-6), 28.4 (C-4 and C-5), 32.2 (C-2 or C-7), 36.4 (C-2 or C-7), 119.0 (C-2′ and C-6′), 122.9 (C-4′), 128.6 (C-3′ and C-5′), 139.3 (C-1′), 169.1 (C-1), 171.2 (C-8)).

### Example 2

### Preparation of Crystalline Composition Consisting Essentially of Suberoylanilide Hydroxamic Acid and Ammonia

SAHA (13.9 g), made in accrodance with the method of Example 1, was dissolved in methanol (556 mL) and heated to 30° C. Then the solution was filtered and allowed to cool down to 25° C. Then it was added to an aqueous ammonia solution (25%, 556 mL) and the resultant system was cooled down to 0-5° C. The suspension was allowed to stir for a further 2h, then filtered and washed with acetone. The desired product, confirmed by PXRD, was obtained as a white solid (10.2 g, 71% yield, ¹H-NMR (DMSO-d6, 300 MHz): 1.20-1.36 (m, 4H, H-4 and H-5), 1.49 (qn, J=7 Hz, 2H, H-3 or H-6), 1.57 (qn, J=7 Hz, H-3 or H-6), 1.94 (t, J=7.3 Hz, 2H, H-2 or H-7), 2.28 (t, J=7.4 Hz, 2H, H-2 or H-7), 6.24 (bs, 2H), 7.01 (t, J=1, 7.4 Hz, 1H, H-4′), 7.27 (t, J=7.9 Hz, 2H, H-3′ and H-5′), 7.58 (d, J=7.6 Hz, 2H, H-2′ and H-6′), 9.84 (s, 1H, NH), ¹³C-NMR (DMSO-d6, 75.5 MHz): 25.0 (C-3 and C-6), 28.4 (C-4 and C-5), 32.2 (C-2 or C-7), 36.4 (C-2 or C-7), 119.0 (C-2′ and C-6′), 122.9 (C-4′), 128.6 (C-3′ and C-5′), 139.3 (C-1′), 169.1 (C-1), 171.2 (C-8)). Mole ratio, 2:1 of SAHA:ammonia, was confirmed by Quantiative

Elemental Analysis according to the values of Table 2.

### Example 3

### Preparation of Suberoylanilide Hydroxamic Acid and Ammonia Crystalline Composition

SAHA (100 mg), made in accrodance with the method of Example 1, was suspended in aqueous ammonia 23% (1 mL) at room temperature for 15 days. The solid isolated was analysed by PXRD, which confirmed the obtainment of the desired product.

### Example 4

### Preparation of Suberoylanilide Hydroxamic Acid and Ammonia Crystalline Composition

SAHA (20 mg), made in accordance with the method of Example 1, was dissolved in ethanol at 70° C. Aqueous ammonia 25% (2 mL) was added and the solution was immediately cooled down to 0° C. A solid precipitated and it was filtered and analysed by PXRD, confirming the obtainment of the desired product.

### Example 5

### Preparation of Suberoylanilide Hydroxamic Acid and Ammonia Crystalline Composition

SAHA (20 mg), made in accrodance with the method of Example 1, was dissolved in isopropanol at 80° C. Aqueous ammonia 25% (2 mL) was added and the solution was immediately cooled down to 0° C. A solid precipitated and it was filtered and analysed by PXRD, confirming the obtainment of the desired product.

### Example 6

### Preparation of Suberoylanilide Hydroxamic Acid and Ammonia Crystalline Composition

SAHA (20 mg), made in accordance with the method of Example 1, was dissolved in a solution 2M of NH₃ in methanol at room temperature. The solution was immediately cooled down to 0° C. A solid precipitated and it was filtered and analysed by PXRD, confirming the obtainment of the desired product.

### Example 7

### Preparation of Suberoylanilide Hydroxamic Acid and Ammonia Crystalline Composition

SAHA (1 g), made in accordance with the method of Example 1, was dissolved in DMSO at room temperature. Aqueous ammonia 25% (10 mL) was added. A solid precipitated and it was filtered and analysed by PXRD, which confirmed the obtainment of the desired product.

### Example 8

### Preparation of Suberoylanilide Hydroxamic Acid and Ammonia Crystalline Composition

SAHA (1 g), made in accordance with the method of Example 1, was dissolved in DMF at room temperature. Aqueous ammonia 25% (10 mL) was added and the solution was immediately cooled down to 0° C. A solid precipitated and it was filtered and analysed by PXRD, which confirmed the obtention of the desired product.

### Example 9

### Preparation of Suberoylanilide Hydroxamic Acid and Ammonia Crystalline Composition

SAHA (1 g), made in accordance with the method of Example 1, was suspended in THF at room temperature. Aqueous ammonia 25% (10 mL) was added. A solid precipitated and it was filtered and analysed by PXRD, which confirmed the obtention of the desired product.

### Example 10

### Preparation of Suberoylanilide Hydroxamic Acid and Ammonia Crystalline Composition

The procedure according to Example 9 was followed, employing in each case a different solvent: dioxane, acetone and acetonitrile. In all cases PXRD analysis confirmed the obtainment of the desired product.

### Example 11

### Preparation of Suberoylanilide Hydroxamic Acid and Ammonia Crystalline Composition

SAHA (20 mg), made in accordance with the method of Example 1, was placed in a 2 mL opened glass vial and then exposed to ammonia vapours inside a 10 mL flask containing 2 mL of aqueous ammonia solution 32%, with no contact between the powder and the liquid; the flask was tightly closed, but no vacuum was operated. After 7 days the obtained solid was analysed by PXRD, which confirmed the obtainment of the desired product.

### Example 12

### Preparation of Crystalline Pure Form α of Suberoylanilide Hydroxamic Acid

A suberoylanilide hydroxamic acid and ammonia crystalline composition (2.52 g), made in accordance with the method of one of examples 2 through 11, was placed on a porcelain capsule and introduced into a preheated (130° C.) oven. The sample was allowed to stand for 90 min, then it was removed and allowed to reach room temperature. Characterization of the obtained solid by PXRD, Table 3, confirmed the complete transformation of the product to crystalline pure and stable Form α of SAHA (2.13 g, 87%, ¹H-NMR (DMSO-d6, 300 MHz): 1.20-1.36 (m, 4H, H-4 and H-5), 1.49 (qn, J=7 Hz, 2H, H-3 or H-6), 1.57 (qn, J=7 Hz, H-3 or H-6), 1.94 (t, J=7.3 Hz, 2H, H-2 or H-7), 2.28 (t, J=7.4 Hz, 2H, H-2 or H-7), 7.01 (t, J=1, 7.4 Hz, 1H, H-4′), 7.27 (t, J=7.9 Hz, 2H, H-3′ and H-5′), 7.58 (d, J=7.6 Hz, 2H, H-2′ and H-6′), 8.68 (bs, 1H, NH), 9.83 (s, 1H, NH), 10.30 (bs, 1H, OH), ¹³C-NMR (DMSO-d6, 75.5 MHz): 25.0 (C-3 and C-6), 28.4 (C-4 and C-5), 32.2 (C-2 or C-7), 36.4 (C-2 or C-7), 119.0 (C-2′ and C-6′), 122.9 (C-4′), 128.6 (C-3′ and C-5′), 139.3 (C-1′), 169.1 (C-1), 171.2 (C-8)).

### Example 13

### Preparation of Crystalline Pure Form α of Suberoylanilide Hydroxamic Acid

A crystalline composition SAHA and ammonia (1.54 g), made in accordance with the method of one of Examples 2 through 11, was added all at once to boiling xylenes (commercial isomers mixture, 50 mL). The suspension was allowed to stir at that temperature for 1 h, then it was cooled down to room temperature, filtered, washed with acetone and dried in the oven at 40° C. under vacuum. PXRD characterisation confirmed the obtention of the desired product.

### Example 14

### Solubility Test Form α Vs Form I

Media
(Solubility mg/mL)

| Crystalline Form | Methanol | HCl 2M |
|---|---|---|
| Form α | 30.00 | 0.19 |
| Form I | 21.80 | 0.10 |

It will be understood that the present invention has been described above by way of examples only. The examples are not intended to limit the scope of the invention. Various modifications and embodiments can be made without departing from the scope and spirit of the invention, which is defined by the following claims only.

1. A crystalline composition comprising suberoylanilide hydroxamic acid (SAHA) and ammonia.

2. The composition of claim 1, wherein the composition has a mole ratio of about 2:1 of SAHA:ammonia.

3. The composition according to claim 1, wherein the composition has a powder X-ray diffraction pattern including characteristic peaks at about 2.4, 4.9, 7.4, 20.9, 21.4, 21.9, 22.2 and 22.7 degrees 2θ, wherein the X-ray diffraction is measured with a Copper X-ray source.

4. The composition according to claim 1, wherein the composition has a powder X-ray diffraction pattern including characteristic peaks at about 2.4, 4.9, 7.4, 20.9, 21.4, 21.9, 22.2 and 22.7 degrees 2θ, wherein the X-ray diffraction is measured with a Copper X-ray source; and having a Differential Scanning calorimetry thermogram substantially similar to that set forth in FIG. 5.

5. A process for preparing a crystalline composition comprising suberoylanilide hydroxamic acid and ammonia, comprising the step of contacting suberoylanilide hydroxamic acid with ammonia until crystal growth is promoted.

6. A process for preparing a suberoylanilide hydroxamic acid and ammonia crystalline composition, comprising:
a) forming a solution of suberoylanilide hydroxamic acid in an organic solvent;
b) promoting crystal growth by adding ammonia; and
c) collecting the crystals.

7. The process according to claim 6, wherein the organic solvent comprises methanol, ethanol, isopropanol, DMF, DMSO or mixtures thereof.

8. The process according to claim 5, wherein the ammonia is added in solution.

9. The process according to claim 5, wherein ammonia is added in an aqueous solution.

10. A pure crystalline Form α of suberoylanilide hydroxamic acid having a powder X-ray diffraction pattern including characteristic peaks at about 9.3, 10.7, 14.5, 14.9, 15.4, 17.7, 19.9, 21.3, 21.5 and 23.3 degrees 2θ, wherein the X-ray diffraction is measured with a Copper X-ray source.

11. The crystalline Form according to claim 10, which further has a DSC thermogram having an endothermic peak at about 162.5±2.0° C.

12. The crystalline Form according to claim 11, which lacks a peak at about 19.2 degrees 2θ.

13. A process for preparing a pure crystalline Form α of suberoylanilide hydroxamic acid, which comprises the step of heating a crystalline composition comprising suberoylanilide hydroxamic acid and ammonia in a range of temperatures of from about 115° C. to about 150° C.

14. The process according to claim 13, which comprises the step of heating a crystalline composition comprising suberoylanilide hydroxamic acid and ammonia in a range of temperatures of from about 120° C. to about 130° C.

15. The product of the process of claim 13.

16. A pharmaceutical composition comprising a therapeutically effective amount of the crystalline Form of claim 10, together with one or more pharmaceutical excipients or carriers.

17. The pharmaceutical composition according to claim 16, wherein the pharmaceutical composition is an oral pharmaceutical composition.

18. The pharmaceutical composition according to claim 16, wherein the pharmaceutical composition has a strength of about 100 mg.

19. A method of treating cutaneous manifestations of cutaneous T-cell lymphoma in a subject who has a progressive, persistent or recurrent disease on or following two systemic therapies, said method comprising the step of administering to the subject an effective amount of the pharmaceutical composition of claim 16.
